(19) United States
(12) Patent Application Publication
(10) Pub. No.: US 2022/0287605 A1
(43) Pub. Date: Sep. 15, 2022

(54) BLOOD PICKER

(71) Applicant: MICROJET TECHNOLOGY CO., LTD., Hsinchu City (TW)

(72) Inventors: Hao-Jan Mou, Hsinchu City (TW); Ching-Sung Lin, Hsinchu City (TW); Yung-Lung Han, Hsinchu City (TW); Chi-Feng Huang, Hsinchu City (TW); Chin-Wen Hsieh, Hsinchu City (TW); Tsung-I Lin, Hsinchu City (TW); Yang Ku, Hsinchu City (TW)

(21) Appl. No.: 17/690,052

(22) Filed: Mar. 9, 2022

(30) Foreign Application Priority Data
Mar. 12, 2021 (TW) 110109027

(51) Int. Cl. A61B 5/15

(52) U.S. Cl. CPC A61B 5/150427; A61B 5/150961; A61B 5/150091; A61B 5/150099

(57) ABSTRACT

A blood picker including one or more needles, a storage device, and a fluid transmission control system is provided. The needle is adapted to be inserted into a blood vessel of a human for blood detection. The storage device is in communication with the needle and provided with a drawing tube in communication with an inner space of the storage device. The fluid transmission control system includes a fluid transmission device, a driving controller, and a power supply. The fluid transmission device is in communication with one end of the drawing tube. The power supply provides a power source for the driving controller to enable the fluid transmission device, so that after the fluid transmission device is enabled, the inner space of the storage device is controlled by the fluid transmission device to generate a pressure difference, thereby allowing the blood to be drawn and stored in the storage device.

### CROSS-REFERENCE TO RELATED APPLICATION

This non-provisional application claims priority under 35 U.S.C. § 119(a) to Patent Application No. 110109027 in Taiwan, R.O.C. on Mar. 12, 2021, the entire contents of which are hereby incorporated by reference.

### BACKGROUND

### Technical Field

The present disclosure relates to a blood picker adapted to draw blood from a human for blood detection.

### Related Art

Owing to developments of medical technologies, more and more information that allows the doctor to identify the disease or the bacterium/virus infecting the patient and determine if the physiological functions of the patient functioning normally can be found from a patient's blood, so as to provide as references of identifying the cause of the disease and determining the type of the medical treatments. Hence, more and more patients go to hospital or health center for drawing the blood in the recent days.

A medical blood drawer known in the relevant art includes a syringe, a needle, and a piston rod. During the operation of blood-drawing, after the medical personnel inserts the needle into the blood-drawing portion of the patient, the medical personnel pulls the piston rod with his/her hand to expand the inner space of the syringe to form a negative pressure environment, thereby providing a negative pressure drawing force for drawing blood from the patient. However, it should be noted that, under such condition, the medical personnel perform the blood drawing operation manually that makes the blood-drawing amount cannot be quantified, and the speed of blood-drawing is not fixed that always causes the patient feel uncomfortable during the operation of blood-drawing. In other words, with the manual blood-drawing operation, the amount and speed of blood-drawing cannot be controlled precisely so as to cause the patient feel uncomfortable easily during the operation of blood-drawing.

In view of above drawbacks, it becomes the main subject of the present invention to design a blood-drawing device by the mechanical blood-drawing operation to replace the traditional manual blood-drawing operation, so as to reduce the discomfort of the patient.

### SUMMARY

The present disclosure provides. One object of the present disclosure is to provide a blood picker comprising a fluid transmission control system to control the inner space of the storage device and to generate a pressure difference therein with respect to the outside environment of the storage device, thereby allowing the needle of the blood picker to draw and store the blood in the storage device as the needle inserted into the blood vessel.

To achieve the above object(s), a general embodiment of the present disclosure provides a blood picker adapted to draw blood from a human for blood detection. The blood picker includes at least one needle, a storage device, and a fluid transmission control system. The at least one needle is adapted to be inserted into a blood vessel of the human for blood detection. The storage device is in communication with the at least one needle, adapted to collect the blood, and provided with a drawing tube in communication with an inner space of the storage device. The fluid transmission control system includes a fluid transmission device, a driving controller, and a power supply. The fluid transmission device is in communication with one end of the drawing tube for drawing the fluid in the inner space of the storage device. The power supply is provided with a power source for the driving controller to enable the fluid transmission device, so that after the fluid transmission device is enabled, the inner space of the storage device is controlled by the fluid transmission device to generate a pressure difference with respect to the outside environment of the storage device, thereby allowing the blood in the blood vessel inserted by the needle to be drawn and stored in the storage device.

### BRIEF DESCRIPTION OF THE DRAWINGS

The disclosure will become more fully understood from the detailed description given herein below, for illustration only and thus not limitative of the disclosure, wherein:

FIG. 1A illustrates a schematic view showing the piston rod is pushed to allow the solid needle portion to be exposed from the hollow soft needle portion according to an exemplary embodiment of the present disclosure;

FIG. 1B illustrates a schematic view showing the piston rod is pulled to allow the solid needle portion to be retracted back into the hollow soft needle portion according to the exemplary embodiment of the present disclosure;

FIG. 1C illustrates an exploded schematic view of the needle and the storage device of the blood picker according to the exemplary embodiment of the present disclosure;

FIG. 1D illustrates a schematic view showing the needle, the storage device, and the fluid transmission control system in combination with each other to perform the operation of blood-drawing according to the exemplary embodiment of the present disclosure;

FIG. 1E illustrates a schematic view showing the needle, the storage device, and the fluid transmission control system in combination with each other to perform the operation of blood-drawing according to another exemplary embodiment of the present disclosure;

FIG. 2A illustrates an exploded view of the components of the gas pump of the blood picker according to the exemplary embodiment of the present disclosure;

FIG. 2B illustrates an exploded view of the components of the gas pump of the blood picker according to the exemplary embodiment of the present disclosure from another view angle;

FIG. 3A illustrates a cross-sectional view of the gas pump of the blood picker according to the exemplary embodiment of the present disclosure;

FIG. 3B illustrates a cross-sectional of the operational step (1) of the gas pump of the blood picker according to the exemplary embodiment of the present disclosure;

FIG. 3C illustrates a cross-sectional of the operational step (2) of the gas pump of the blood picker according to the exemplary embodiment of the present disclosure;

FIG. 3D illustrates a cross-sectional of the operational step (3) of the gas pump of the blood picker according to the exemplary embodiment of the present disclosure;

FIG. 4A illustrates a perspective view of the box gas pump of the blood picker according to the exemplary embodiment of the present disclosure;

FIG. 4B illustrates an exploded view of the box gas pump of the blood picker according to the exemplary embodiment of the present disclosure;

FIG. 4C illustrates an exploded view of the box gas pump of the blood picker according to the exemplary embodiment of the present disclosure from another view angle;

FIG. 5A illustrates a cross-sectional view of the box gas pump of the blood picker according to the exemplary embodiment of the present disclosure;

FIG. 5B illustrates a cross-sectional of the operational step (1) of the box gas pump of the blood picker according to the exemplary embodiment of the present disclosure;

FIG. 5C illustrates a cross-sectional of the operational step (2) of the box gas pump of the blood picker according to the exemplary embodiment of the present disclosure;

FIG. 6 illustrates a cross-sectional view of the microelectromechanical systems (MEMS) pump of the blood picker according to the exemplary embodiment of the present disclosure;

FIG. 7A illustrates a cross-sectional of the operational step (1) of the MEMS pump of the blood picker according to the exemplary embodiment of the present disclosure;

FIG. 7B illustrates a cross-sectional of the operational step (2) of the MEMS pump of the blood picker according to the exemplary embodiment of the present disclosure; and

FIG. 7C illustrates a cross-sectional of the operational step (3) of the MEMS pump of the blood picker according to the exemplary embodiment of the present disclosure.

### DETAILED DESCRIPTION

The present disclosure will now be described more specifically with reference to the following embodiments. It is to be noted that the following descriptions of different embodiments of this disclosure are presented herein for purpose of illustration and description only, and it is not intended to limit the scope of the present disclosure.

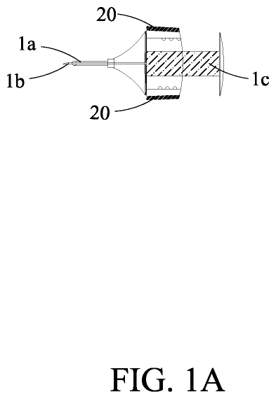

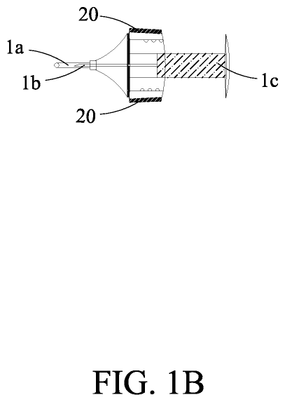

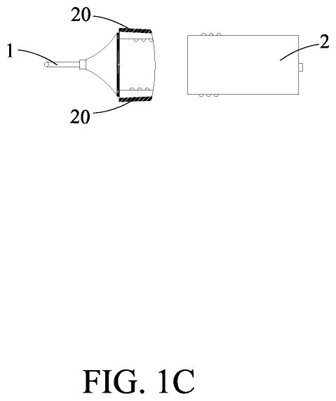

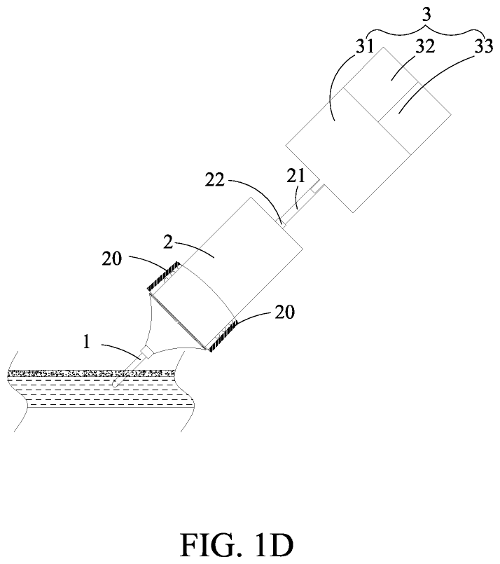

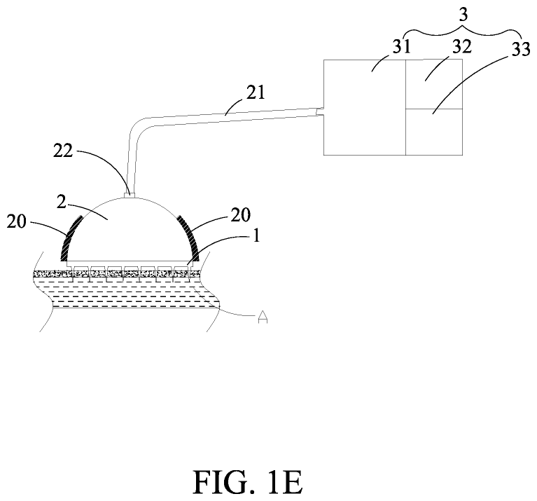

Please refer to FIG. 1A to FIG. 1D. A first preferred embodiment of the present disclosure provides a blood picker adapted to draw blood from a human for blood detection including at least one needle 1, a storage device 2, and a fluid transmission control system 3.

The needle 1 is adapted to be inserted into a blood vessel A of the human for blood detection. The needle 1 includes a hollow soft needle portion 1a, a solid needle portion 1b, and a piston rod 1c. The solid needle portion 1b is enclosed by the hollow soft needle portion 1a. The solid needle portion 1b is received in the hollow soft needle portion 1a and slightly exposed from the hollow soft needle portion 1a. The piston rod 1c is connected to the solid needle portion 1b. During the operation of blood-drawing, the piston rod 1c is pushed to allow the solid needle portion 1b to penetrate into the blood vessel A of the human, so that the hollow soft needle portion 1a is also inserted into the blood vessel A. Then, the piston rod 1c is pulled to drive the solid needle portion 1b to be removed out of the blood vessel A, thereby leaving the hollow soft needle portion 1a in the blood vessel A of the human. In one embodiment, the length of the hollow soft needle portion 1a is in a range between 1000 μm and 2000 μm, and the hole diameter of the hollow soft needle portion 1a is in a range between 10 μm and 1000 μm.

The storage device 2 is in communication with the needle 1 for storing and collecting blood. The storage device 2 is provided with a drawing tube 21, a connection portion 22, and a blood detector 20. The blood detector 20 is disposed outside the storage device 2. The connection portion 22 is detachably connected between the storage device 2 and the drawing tube 21, so that the storage device 2 and the drawing tube 21 can be assembled with or detached from each other through the connection portion 22. The blood detector 20 is adapted to detect whether blood exists in the storage device 2 so as to determine whether the needle 1 is correctly inserted into the blood vessel A. The drawing tube 21 is in communication with an inner space of the storage device 2.

The fluid transmission control system 3 includes a fluid transmission device 31, a driving controller 32, and a power supply 33. The fluid transmission device 31 is in communication with one end of the drawing tube 21 and is enabled to draw the fluid in the inner space of the storage device 2. The power supply 33 is provided with a power source for the driving controller 32 to enable the fluid transmission device 31. Therefore, after the fluid transmission device 31 is enabled, the inner space of the storage device 2 is controlled by the fluid transmission device 31 to generate a pressure difference with respect to the outside environment of the storage device 2, thereby allowing the blood in the blood vessel A inserted by the needle 1 to be drawn and stored in the storage device 2.

In this embodiment, the needle 1, the storage device 2, and the drawing tube 21 are disposable. Moreover, the connection part between the fluid transmission control system 3 and the drawing tube 21 may be provided with a non-return valve (not shown). Therefore, the blood can be prevented from reflowing back to the fluid transmission control system 3 through the non-return valve, thereby the fluid transmission control system 3 can be utilized repeatedly.

Please further refer to FIG. 1E. A second preferred embodiment of the present disclosure provides a blood picker adapted to draw blood from a human for blood detection including a plurality of microneedles 1, a storage device 2, and a fluid transmission control system 3.

The microneedles 1 are adapted to be inserted into a blood vessel A of the human for blood detection. In one embodiment, the length of the microneedles 1 is in a range between 1000 μm and 2000 μm, and the hole diameter of the microneedles 1 is in a range between 10 μm and 1000 μm.

The storage device 2 is in communication with the microneedles 1 for storing and collecting blood. The storage device 2 is provided with a drawing tube 21, a connection portion 22, and a blood detector 20. The blood detector 20 is disposed outside the storage device 2. The connection portion 22 is detachably connected between the storage device 2 and the drawing tube 21, so that the storage device 2 and the drawing tube 21 can be assembled with or detached from each other through the connection portion 22. The blood detector 20 is adapted to detect whether blood exists in the storage device 2 so as to determine whether the needle 1 is correctly inserted into the blood vessel A. The drawing tube 21 is in communication with an inner space of the storage device 2.

The fluid transmission control system 3 includes a fluid transmission device 31, a driving controller 32, and a power supply 33. The fluid transmission device 31 is in communication with one end of the drawing tube 21 and is enabled to draw the fluid in the inner space of the storage device 2. The power supply 33 provides a power source for the driving controller 32 to enable the fluid transmission device 31. Therefore, after the fluid transmission device 31 is enabled, the inner space of the storage device 2 is controlled by the fluid transmission device 31 to generate a pressure difference with respect to the outside environment of the storage device 2, thereby allowing the blood in the blood vessel A inserted by the needles 1 to be drawn and stored in the storage device 2.

In this embodiment, the microneedles 1, the storage device 2, and the drawing tube 21 are disposable. Moreover, the connection part between the fluid transmission control system 3 and the drawing tube 21 may be provided with a non-return valve (not shown). Therefore, the blood can be prevented from reflowing back to the fluid transmission control system 3 through the non-return valve, thereby the fluid transmission control system 3 can be utilized repeatedly.

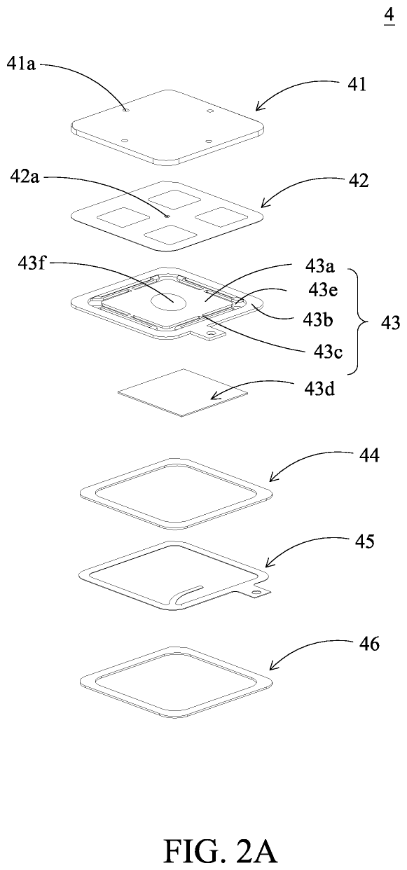

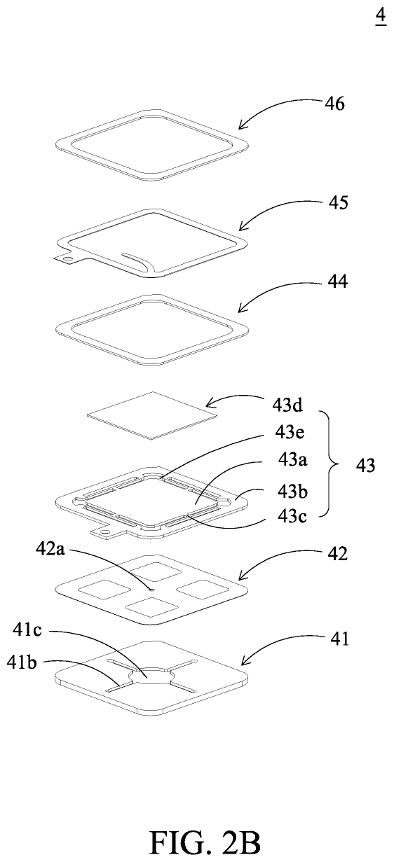

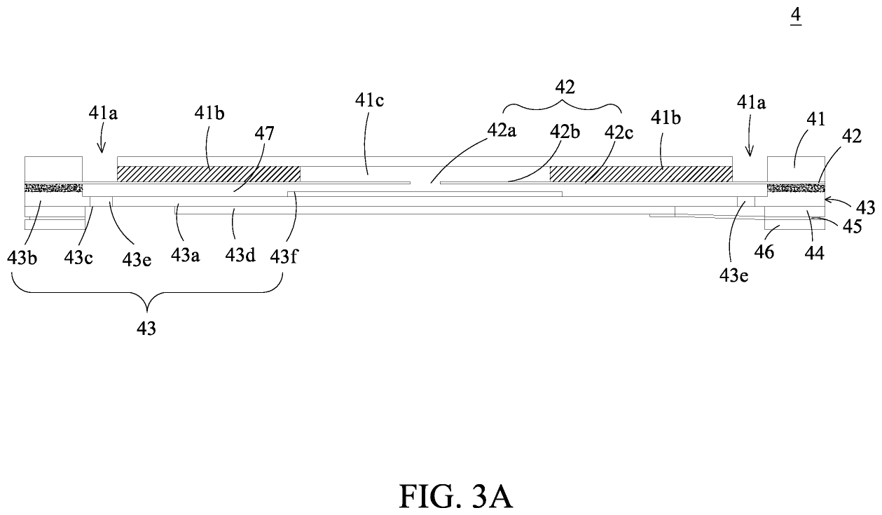

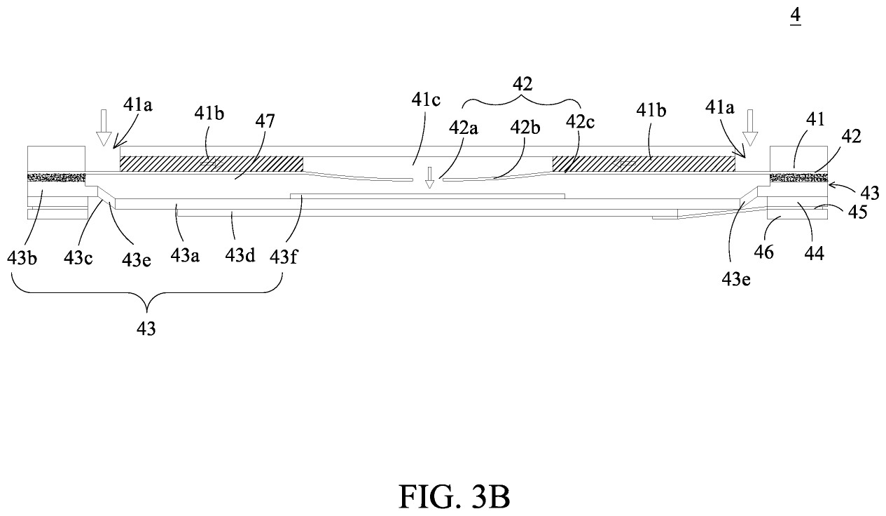

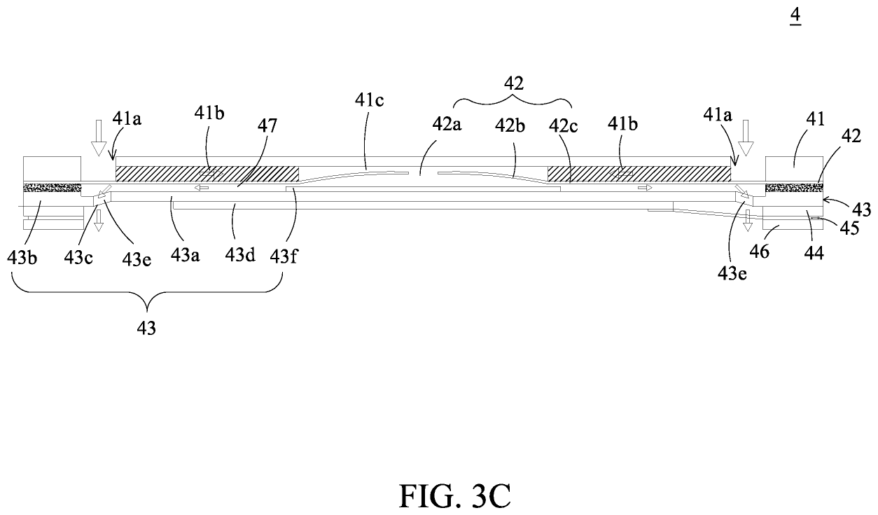

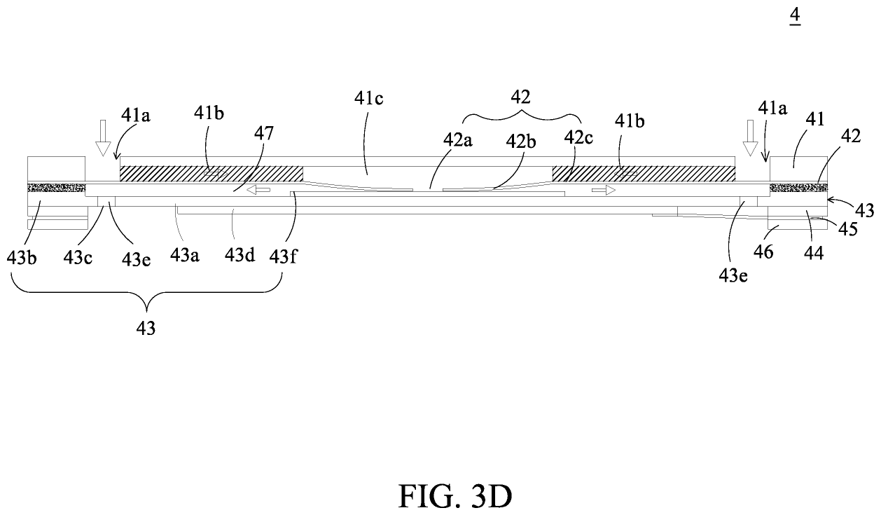

Please refer to FIG. 2A to FIG. 3D. In this embodiment, the fluid transmission device 31 is a gas pump 4, and the gas pump 4 includes an inlet plate 41, a resonance sheet 42, a piezoelectric actuator 43, a first insulation sheet 44, a conductive sheet 45, and a second insulation sheet 46 sequentially stacked with each other. The inlet plate 41 has at least one inlet hole 41a, at least one convergence channel 41b, and a convergence chamber 41c. The inlet hole 41a is adapted to introduce the gas outside the gas pump 4 into the gas pump 4. The inlet hole 41a correspondingly penetrates into the convergence channel 41b, and the convergence channel 41b is converged at the convergence chamber 41c, so that the gas introduced from the inlet hole 41a can be converged at the convergence chamber 41c. In this embodiment, the number of the inlet holes 41a and the number of the convergence channels 41b are the same. Moreover, in this embodiment, the number of the inlet holes 41a and the number of the convergence channels 41b both are four, respectively, but not limited thereto. The four inlet holes 41a are respectively in communication with the four convergence channels 41b, and the four convergence channels 41b are converged into the convergence chamber 41c.

In this embodiment, the resonance sheet 42 may be attached and assembled on the inlet plate 41. Furthermore, the resonance sheet 42 has a perforation 42a, a movable portion 42b, and a fixed portion 42c. The perforation 42a is located at a center portion of the resonance sheet 42 and is corresponding to the convergence chamber 41c of the inlet plate 41. The movable portion 42b is disposed at a portion surrounding the perforation 42a that is corresponding to the convergence chamber 41c. The fixed portion 42c is disposed at an outer periphery of the resonance sheet 42 and is attached to the inlet plate 41.

In this embodiment, the piezoelectric actuator 43 is attached and disposed in corresponding to the resonance sheet 42 and includes a suspension plate 43a, an outer frame 43b, at least one supporting element 43c, a piezoelectric element 43d, at least one gap 43e, and a protruding portion 43f. In the embodiments of the present disclosure, the suspension plate 43a is in square shape. It is understood that, the reason why the suspension plate 43a adopts the square shape is that, comparing with a circle suspension plate having a diameter equal to the side length of the square suspension plate 43a, the square suspension plate 43a has an advantage of saving electricity. The power consumption of a capacitive load operated under a resonance frequency may increase as the resonance frequency increases, and since the resonance frequency of a square suspension plate 43a is much lower than that of a circular suspension plate, the power consumption of the square suspension plate 43a is relatively low as well. Consequently, the square design of the suspension plate 43a used in one or some embodiments of the present disclosure has the benefit of power saving. In the embodiments of the present disclosure, the outer frame 43b is disposed around the periphery of the suspension plate 43a. The at least one supporting element 43c is connected between the suspension plate 43a and the outer frame 43b to provide a flexible support for the suspension plate 43a. In the embodiments of the present disclosure, the piezoelectric element 43d has a side length, which is shorter than or equal to a side length of the suspension plate 43a. The piezoelectric element 43d is attached to a surface of the suspension plate 43a so as to drive the suspension plate 43a to bend and vibrate when the piezoelectric element 43d is applied with a voltage. There is at least one gap 43e formed between the suspension plate 43a, the outer frame 43b, and the at least one supporting element 43c for the gas to flow therethrough. The protruding portion 43f is disposed on a surface of the suspension plate 43a opposite to the surface of the suspension plate 43a where the piezoelectric element 43d is attached. In this embodiment, the protruding portion 43f may be a convex structure protruding out from and integrally formed with the surface of the suspension plate 43a opposite to the surface of the suspension plate 43a where the piezoelectric element 43d is attached by performing an etching process on the suspension plate 43a.

In this embodiment, the inlet plate 41, the resonance sheet 42, the piezoelectric actuator 43, the first insulation plate 44, the conductive plate 45, and the second insulation plate 46 are stacked sequentially with each other, and a chamber space 47 is formed between the suspension plate 43a of the piezoelectric actuator 43 and the resonance sheet 42. The chamber space 47 can be formed by filling a material, such as conductive adhesive, at the gap between the resonance sheet 42 and the outer frame 43b of the piezoelectric actuator 43, but not limited thereto, so as to maintain a certain distance between the resonance sheet 42 and the suspension plate 43a to allow the gas to be guided and flow more quickly. Further, since an appropriate distance is maintained between the suspension plate 43a and the resonance sheet 42, the interference raised from the contact between the suspension plate 43a and the resonance sheet 42 can be reduced, so that the noise generated can be reduced as well. In other embodiments, the required thickness of the conductive adhesive between the resonance sheet 42 and the outer frame 43b of the piezoelectric actuator 43 can be decreased by increasing the height of the outer frame 43b of the piezoelectric actuator 43. Accordingly, the entire structure of the gas pump 4 would not be indirectly affected by the hot pressing temperature and the cooling temperature owing to the filling material of conductive adhesive, thereby avoiding the actual spacing of the chamber space 47 from being affected by the thermal expansion and contraction of the filling material of the conductive adhesive, but not limited thereto. Moreover, the height of the chamber space 47 also affects the transmission efficiency of the gas pump 4. Therefore, it is important to maintain a fixed height of the chamber space 47 for the purpose of achieving stable transmission efficiency of the gas pump 4.

In order to understand the operation steps in transmitting gas of the aforementioned gas pump 4, please refer to FIG. 3B to FIG. 3D. The piezoelectric element 43d of the piezoelectric actuator 43 deforms after being applied with a driving voltage, and the piezoelectric element 43d drives the suspension plate 431 to move downwardly and to move away from the inlet plate 41. Thus, the volume of the chamber space 47 is increased to generate a negative pressure inside the chamber space 47, thereby drawing the gas in the convergence chamber 41c into the chamber space 47. At the same time, owing to the resonance effect, the resonance sheet 42 moves downwardly and moves away from the inlet plate 41, and thus increases the volume of the convergence chamber 41c. Furthermore, since the gas inside the convergence chamber 41c is drawn into the chamber space 47, the convergence chamber 41c is in a negative pressure state, and the gas can be drawn into the convergence chamber 41c. The piezoelectric element 43d drives the suspension plate 431 to move upwardly and to move toward the inlet plate 41 and compresses the chamber space 47. Similarly, since the resonance sheet 42 resonates with the suspension plate 431, the resonance sheet 42 also moves upwardly toward the inlet plate 41, thereby pushing the gas in the chamber space 47 to move downwardly and be transmitted out of the gas pump 4 through the at least one gap 43e so as to achieve the effect of gas transmission. When the suspension plate 43a moves resiliently to its original position, the resonance sheet 42 still moves downwardly away from the inlet plate 43a due to its inertia momentum. At this time, the resonance sheet 42 compresses the chamber space 47, so that the gas in the chamber space 47 is moved toward the gap 43e and the volume of the convergence chamber 41c is increased. Accordingly, the gas can be drawn into the convergence chamber 41c continuously through the inlet holes 41a and the convergence channels 41b and then converged at the convergence chamber 41c. Through continuously repeating the foregoing operation steps, the gas pump 4 can make the gas continuously enter into the flow paths formed by the inlet plate 41 and the resonance sheet 42 from the inlet holes 41a, thereby generating a pressure gradient. The gas is then transmitted downwardly through the gap 43e. As a result, the gas can flow at a relatively high speed, thereby achieving the effect of gas transmission of the gas pump 4.

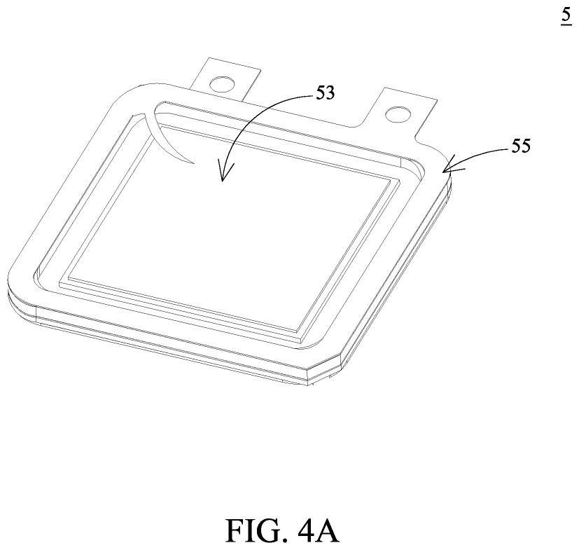

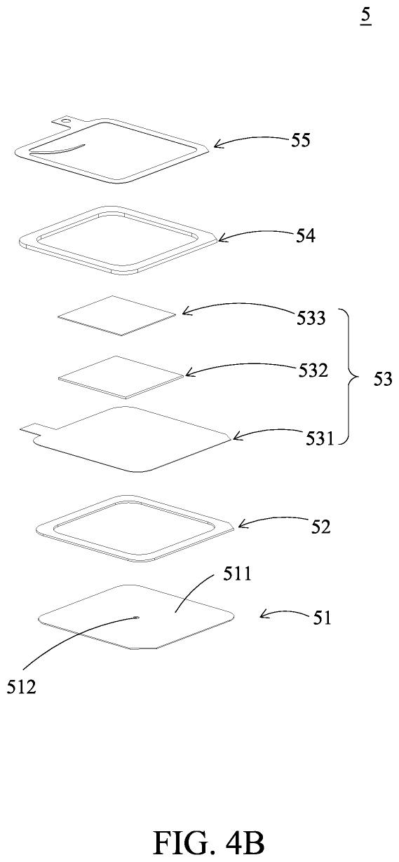

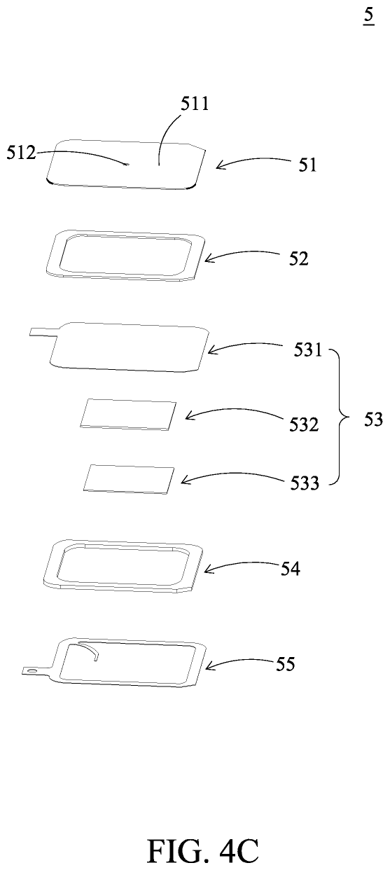

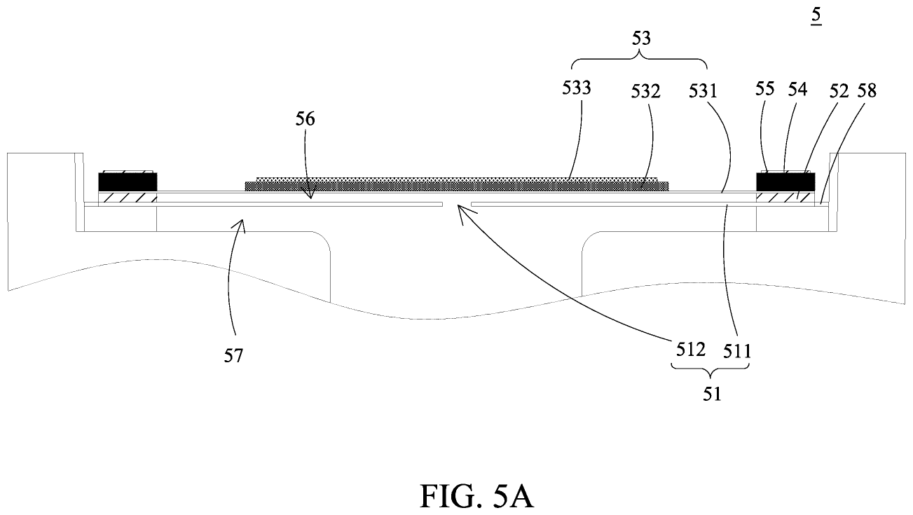

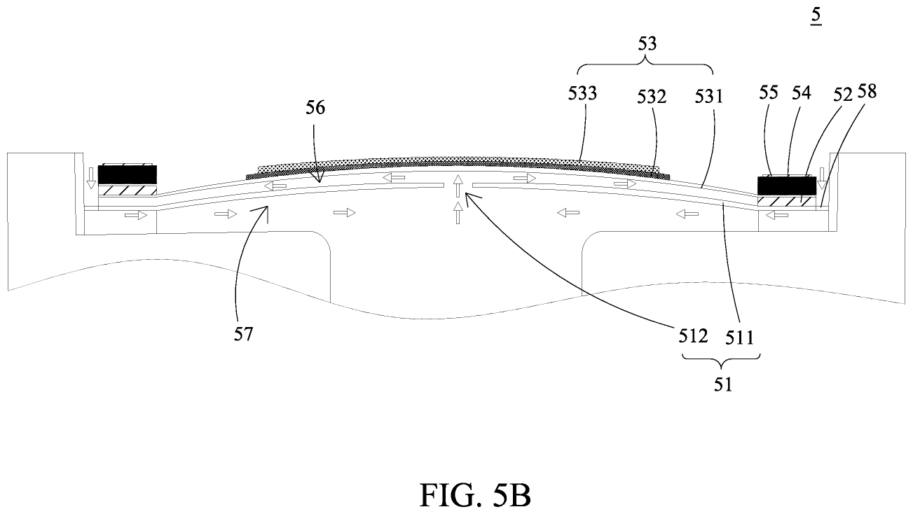

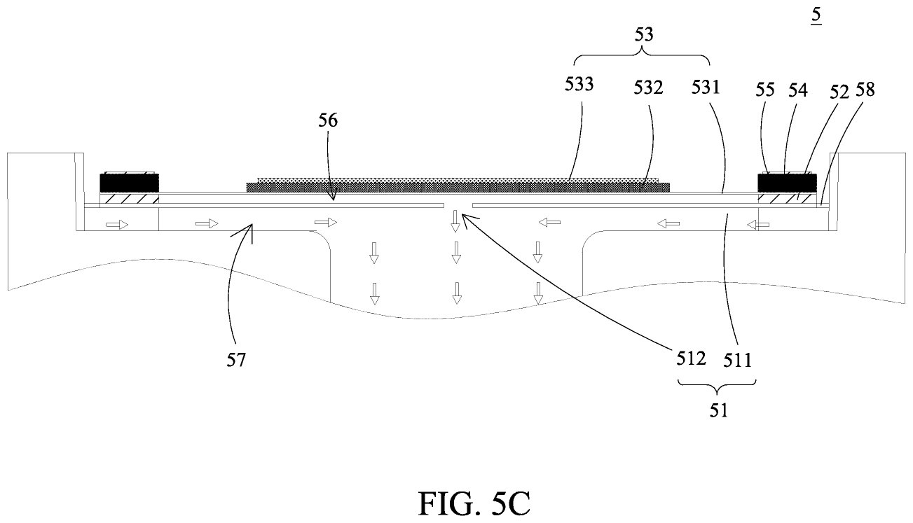

Please refer to FIG. 4A to FIG. 5C. In this embodiment, the fluid transmission device 31 is a box gas pump 5, and the box gas pump 5 includes a nozzle plate 51, a chamber frame 52, an actuation body 53, an insulation frame 54, and a conductive frame 55. The nozzle plate 51 includes a suspension sheet 511 and a central hole 512. The suspension sheet 511 is able to bend and vibrate. The central hole 512 is formed at a center portion of the suspension sheet 511. The chamber frame 52 is carried and stacked on the suspension sheet 511. The actuation body 53 includes a piezoelectric carrier plate 531, an adjusting resonance plate 532, and a piezoelectric plate 533 sequentially stacked with each other. The piezoelectric carrier plate 531 is carried and stacked on the chamber frame 52, and the piezoelectric carrier plate 531 is provided for bending and vibrating reciprocatingly when the piezoelectric carrier plate 531 is applied with a voltage. The insulation frame 54 is carried and stacked on the piezoelectric carrier plate 531 of the actuation body 53, and the conductive frame 55 is carried and stacked on the insulation frame 54. Therefore, the nozzle plate 51 is fixedly disposed and positioned to define a gap 58 at a periphery of the nozzle plate 51 for gas flowing therethrough, a gas flow chamber 57 is formed at the bottom of the nozzle plate 51, and a resonance chamber 56 is formed between the actuation body 53, the chamber frame 52, and the suspension sheet 511. The nozzle plate 51 is driven through driving the actuation body 53 to generate resonance effect, so that the suspension sheet 511 of the nozzle plate 51 shifts and vibrates reciprocatingly, and thus drawing the gas entering into the gas flow chamber 57 through the gap 58 and then discharging out of the gas flow chamber 57, thereby achieving fluid transmission.

When the piezoelectric plate 533 drives the suspension sheet 511 of the nozzle plate 51 to move in a direction away from the bottom surface, the suspension sheet 511 of the nozzle plate 51 is driven to move in the direction away from the bottom surface of the positioning bump correspondingly. Hence, the volume of the gas flow chamber 57 expands dramatically, so that the internal pressure of the gas flow chamber 57 decreases and creates a negative pressure, thereby drawing the gas outside the box gas pump 5 to flow into the box gas pump 5 through the gaps 58 and enter into the resonance chamber 56 through the central hole 512, and thus increasing the gas pressure of the resonance chamber 56 and generating a pressure gradient. Furthermore, as shown in FIG. 5C, when the piezoelectric plate 533 drives the suspension sheet 511 of the nozzle plate 51 to move toward the bottom surface, the gas inside the resonance chamber 56 is pushed to flow out quickly through the central hole 512 so as to further squeeze the fluid inside the gas flow chamber 57, thereby the converged fluid can be quickly and massively ejected out in a state closing to an ideal fluid state under the Benulli's law. Furthermore, after the gas is discharged out of the resonance chamber 56, the internal pressure of the resonance chamber 56 is lower than the equilibrium pressure due to the inertia, as a result, the pressure difference guides the gas outside the resonance chamber 56 into the resonance chamber 56 again. Therefore, through repeating the foregoing steps, the piezoelectric plate 533 is able to bend and vibrate reciprocatingly. Through controlling the vibration frequency of the gas inside the resonance chamber 56 to be close the vibration frequency of the piezoelectric plate 533 and generate the Helmholtz resonance effect, high-speed and large-volume fluid transmission can be achieved.

Therefore, the nozzle plate 51 is fixedly disposed and positioned to define a gap at a periphery of the nozzle plate 51 for gas flowing therethrough, a gas flow chamber 57 is formed at the bottom of the nozzle plate 51, and a resonance chamber 56 is formed between the actuation body 53, the chamber frame 52, and the suspension sheet 511. The nozzle plate 51 is driven through driving the actuation body 53 to generate resonance effect, so that the suspension sheet 511 of the nozzle plate 51 shifts and vibrates reciprocatingly, and thus drawing the gas entering into the gas flow chamber 57 and then discharging out of the gas flow chamber 57.

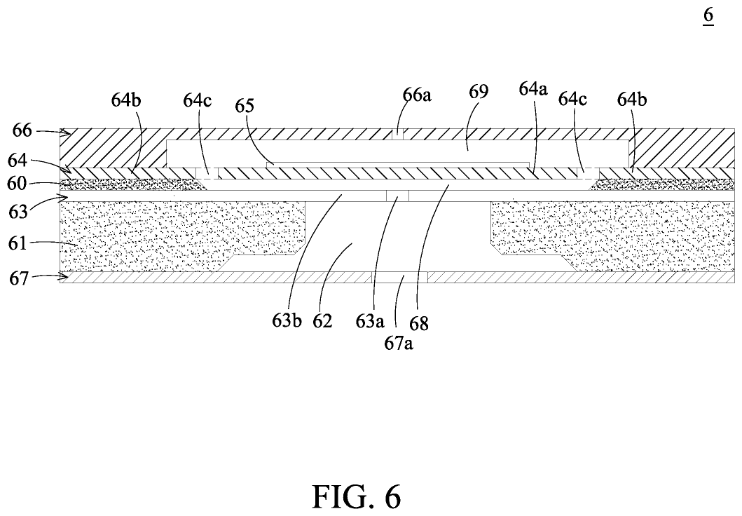

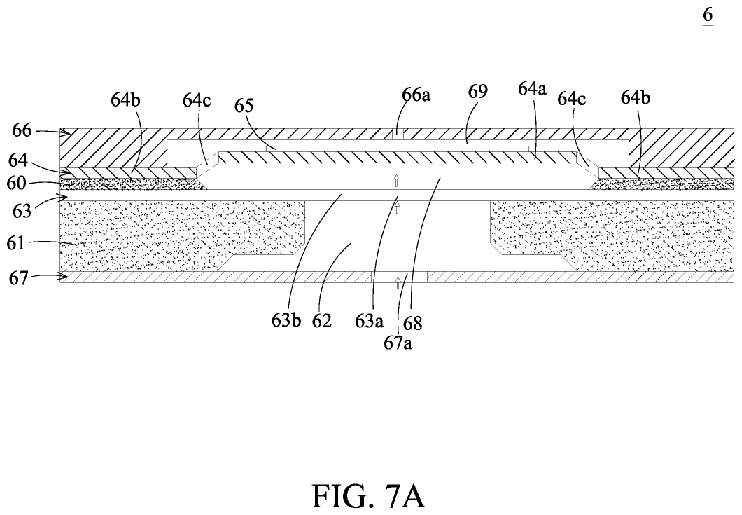

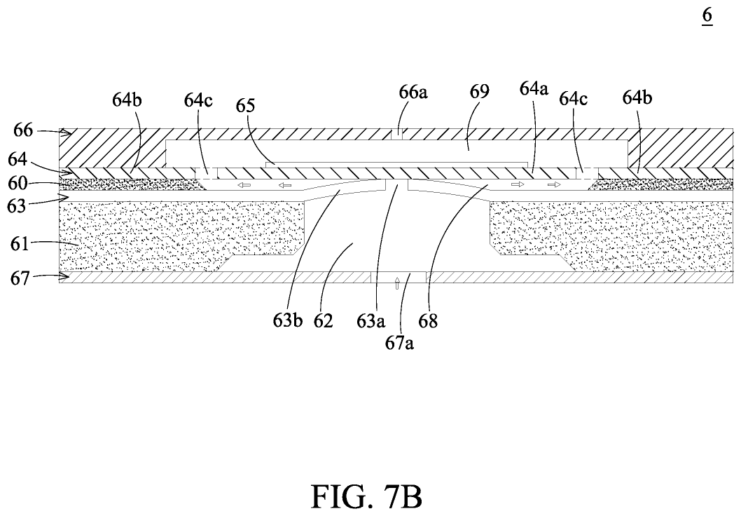

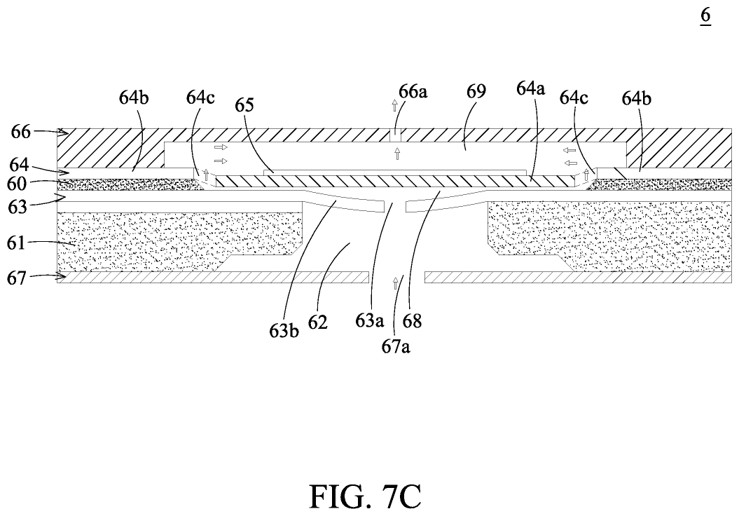

Please refer to FIG. 6 to FIG. 7C. In this embodiment, the fluid transmission device 31 is a microelectromechanical systems (MEMS) pump 6 manufactured through semiconductor processes. The MEMS pump 6 includes a substrate 61, a first chamber 62, a resonance plate 63, an actuation plate 64, a piezoelectric element 65, an outlet board 66, and an inlet board 67. The first chamber 62 is formed on the substrate 61 by etching. A hollow hole 63a and a movable portion 63b are formed on the resonance board 63 by etching, and the resonance board 63 is stacked on the substrate 61. The movable portion 63b is a flexible structure formed by a portion of the resonance board 63 which is not fixedly disposed on the substrate 61. A spacing layer 60 is coated and stacked on portions of the resonance board 63 except the movable portion 63b. A suspension portion 64a, an outer frame portion 64b, and a plurality of brackets 64c are formed on the actuation board 64 by etching, and the actuation board 64 is stacked on the spacing layer 60. The suspension portion 64a is connected to and suspended from the outer frame portion 64b through the brackets 64c, and the suspension portion 64a is supported by the outer frame portion 64b through the brackets 64c. A plurality of through holes is provided between the suspension portion 64a, the outer frame portion 64b, and the brackets 64c for gas flowing therethrough. A second chamber 68 is defined between the actuation board 64 and the resonance board 63. The piezoelectric element 65 is coated and stacked on the suspension portion 64a of the actuation board 64. The outlet board 66 is provided with a third chamber 69 and an outlet hole 66a by etching, and the outlet board 66 is stacked on the outer frame portion 64b of the actuation board 64, so that the third chamber 69 is corresponding to the suspension portion 64a and a part of the outer frame portion 64b of the actuation board 64. The outlet hole 66a is in communication with the third chamber 69. The inlet board 67 is provided with at least one inlet hole 67a by etching, and the inlet board 67 is stacked below the substrate 61. Therefore, in the fluid transmission device 31, the actuation board 64 is driven by the piezoelectric element 65 to move reciprocatingly so as to draw the fluid into the first chamber 62 through the inlet hole 67a and pass through the hollow hole 63a of the resonance board 63, thereby a resonance effect is generated between the actuation board 64 and the movable portion 63b of the resonance board 63 for gas transmission, thereby achieving fluid transmission.

In the case that the fluid transmission device 31 is not enabled (that is, in its initial state), when the piezoelectric element 65 is applied with a voltage, the piezoelectric element 65 is deformed to drive the actuation board 64 to move reciprocatingly along a vertical direction, thereby increasing the volume of the second chamber 68 and reducing the pressure in the second chamber 68 as the suspension portion 64a of the actuation board 64 is vibrated upwardly. Therefore, the fluid enters the fluid transmission device 31 from the inlet hole 67a of the inlet board 67 and is converged at the first chamber 62 and flows upwardly into the second chamber 68 through the hollow hole 63a on the resonance board 63 corresponding to the first chamber 62. Next, the vibration of the suspension portion 64a of the actuation board 64 drives the resonance board 63 to generate resonance effect, so that the movable portion 63b vibrates upwardly as the suspension of the actuation board 64a vibrates downwardly, thus resulting in that the movable portion 63b of the resonance board 63 is attached below the suspension portion 64a of the actuation board 64. At this moment, the gap between the hollow hole 63a of the resonance board 63 and the second chamber 68 is sealed. Therefore, the second chamber 68 is compressed, so that the volume of the second chamber 68 is decreased and the pressure in the second chamber 68 is increased, while the volume of the third chamber 69 is increased and the pressure in the third chamber 69 is decreased, thereby creating a pressure gradient in the fluid transmission device 31. Thus, the fluid in the second chamber 68 is compressed to flow toward two sides of the second chamber 68 and then flows into the third chamber 69 through the through holes 64c of the actuation board 64. The suspension portion 64a of the actuation board 64 keeps vibrating downwardly to drive the movable portion 63b of the resonance board 63 to move downwardly, thereby allowing the second chamber 68 to be further compressed and most of the fluid in the second chamber 68 flows to the third chamber 69 for temporary storage. Under such operations repeatedly, the fluid can be drawn into the fluid transmission device 31 from the inlet hole 67a and can be discharged outwardly from the outlet hole 66a, thereby achieving fluid transmission.

Accordingly, in this embodiment of the present disclosure, the piezoelectric element 65 drives the actuation board 64 to shift and vibrate reciprocatingly so as to draw the gas into the first chamber 62 through the inlet hole 67a and pass through the hollow hole 63a of the resonance board 63, so that a resonance effect is generated between the actuation board 64 and the movable portion 63b of the resonance board 63 for gas transmission.

Based on the above description, in the blood picker according to one or some embodiments of the present disclosure, the fluid transmission control system 3 is provided for controlling the flowing rate and the flow amount of the blood during the operation of blood-drawing. Hence, the operation of blood-drawing and blood-collection can be achieved by using a mechanical structure. Moreover, the storage device 2 for storing the blood is designed as a combinable and detachable component so as to allow the storage device 2 filled with the blood can be detached and delivered to the specimen collection station for the medical personnel. Accordingly, the blood picker according to one or some embodiments of the present disclosure not only can properly solve the problems of failing to maintain the amount and speed of blood-drawing in the traditional manual blood-drawing operation, but also can effectively reduce the discomforts of the patient derived from the operation of blood-drawing.

What is claimed is:

1. A blood picker adapted to draw blood from a human for blood detection, wherein the blood picker comprises:
at least one needle adapted to be inserted into a blood vessel of the human for blood detection;
a storage device in communication with the at least one needle, wherein the storage device is adapted to collect the blood and provided with a drawing tube in communication with an inner space of the storage device; and
a fluid transmission control system comprising a fluid transmission device, a driving controller, and a power supply, wherein the fluid transmission device is in communication with one end of the drawing tube for drawing the fluid in the inner space of the storage device; and the power supply is provided with a power source for the driving controller to enable the fluid transmission device, so that after the fluid transmission device is enabled, the inner space of the storage device is controlled by the fluid transmission device to generate a pressure difference with respect to the outside environment of the storage device, thereby allowing the blood in the blood vessel inserted by the needle to be drawn and stored in the storage device.

2. The blood picker according to claim 1, wherein the needle comprises a hollow soft needle portion and a solid needle portion; the solid needle portion is received in the hollow soft needle portion, slightly exposed from the hollow soft needle portion, and adapted to penetrate the blood vessel of the human, so that the hollow soft needle portion is allowed to be inserted into the blood vessel; after the solid needle portion is removed from the hollow soft needle portion, the hollow soft needle portion is adapted to be connected to the storage device.

3. The blood picker according to claim 2, wherein a length of the hollow soft needle portion is in a range between 1000 μm and 2000 μm, and a hole diameter of the hollow soft needle portion is in a range between 10 μm and 1000 μm.

4. The blood picker according to claim 1, wherein the needle is a microneedle.

5. The blood picker according to claim 4, wherein the blood picker comprises a plurality of microneedles; the microneedles are integrally disposed at one side of the storage device, and the microneedles are adapted to be inserted into the blood vessel of the human for blood detection.

6. The blood picker according to claim 5, wherein a length of each of the microneedles is in a range between 1000 μm and 2000 μm, and a hole diameter of each of the microneedles is in a range between 10 μm and 1000 μm.

7. The blood picker according to claim 1, wherein a blood detector is disposed outside the storage device, and the blood detector is adapted to detect whether blood exists in the storage device so as to determine whether the needle is correctly inserted into the blood vessel.

8. The blood picker according to claim 1, wherein a connection portion is provided between the storage device and the drawing tube, and the connection portion is adapted to allow the storage device and the drawing tube to be assembled with or detached from each other.

9. The blood picker according to claim 1, wherein the needle, the storage device, and the drawing tube are disposable.

10. The blood picker according to claim 1, wherein the fluid transmission device of the fluid transmission control system is a gas pump, and the gas pump comprises:
an inlet plate having at least one inlet hole, at least one convergence channel, and a convergence chamber, wherein the at least one inlet hole is used to introduce the gas into the gas pump, the at least one inlet hole correspondingly penetrates into the at least one convergence channel, and the at least one convergence channel is converged at the convergence chamber, so that the gas introduced from the at least one inlet hole is converged at the convergence chamber;
a resonance sheet attached to the inlet plate, wherein the resonance sheet has a perforation, a movable portion, and a fixed portion, wherein the perforation is located at a center portion of the resonance sheet and is corresponding to the convergence chamber of the inlet plate, the movable portion is disposed at a portion surrounding the perforation that is corresponding to the convergence chamber, and the fixed portion is disposed at an outer periphery of the resonance sheet and is attached to the inlet plate; and
a piezoelectric actuator attached and disposed in corresponding to the resonance sheet, wherein the piezoelectric actuator comprises a suspension plate, an outer frame, at least one supporting element, and a piezoelectric element; the suspension plate is capable of bending and vibrating; the outer frame is disposed around a periphery of the suspension plate; the at least one supporting element is formed between the suspension plate and the outer frame to provide a flexible support for the suspension plate; the piezoelectric element is attached to a surface of the suspension plate so as to drive the suspension plate to bend and vibrate when the piezoelectric element is applied with a voltage;
wherein a chamber space is formed between the resonance sheet and the piezoelectric actuator, so that when the piezoelectric actuator is driven by the voltage, the gas outside the gas pump is introduced into the gas pump through the at least one inlet hole of the inlet plate, is converged at the convergence chamber through the at least one convergence channel, flows through the perforation of the resonance sheet, and is transmitted by a resonance effect resulting between the piezoelectric actuator and the movable portion of the resonance sheet.

11. The blood picker according to claim 10, wherein the gas pump further comprises a first insulation sheet, a conductive sheet, and a second insulation sheet; and the inlet plate, the resonance sheet, the piezoelectric actuator, the first insulation sheet, the conductive sheet, and the second insulation sheet are sequentially stacked with each other.

12. The blood picker according to claim 1, wherein the fluid transmission device of the fluid transmission control system is a box gas pump, and the box gas pump comprises:
a nozzle plate comprising a suspension sheet and a central hole, wherein the suspension sheet is capable of bending and vibrating, and the central hole is formed at a center portion of the suspension sheet;
a chamber frame carried and stacked on the suspension sheet;
an actuation body carried and stacked on the chamber frame comprising a piezoelectric carrier plate, an adjusting resonance plate, and a piezoelectric plate sequentially stacked with each other, wherein the actuation body is provided for bending and vibrating reciprocatingly when the actuation body is applied with a voltage;
an insulation frame carried and stacked on the piezoelectric carrier plate of the actuation body; and
a conductive frame carried and stacked on the insulation frame;
wherein the nozzle plate is fixedly disposed and positioned so as to define a gap at a periphery of the nozzle plate for gas flowing therethrough, a gas flow chamber is formed at a bottom of the nozzle plate, and a resonance chamber is formed between the actuation body, the chamber frame, and the suspension sheet, wherein the nozzle plate is driven through driving the actuation body by the voltage, so that the suspension sheet of the nozzle plate shifts and vibrates reciprocatingly, and thus drawing the gas entering into the gas flow chamber through the gap and then discharging out of the gas flow chamber.

13. The blood picker according to claim 1, wherein the fluid transmission device of the fluid transmission control system is a microelectromechanical systems (MEMS) pump manufactured through semiconductor processes, and the MEMS pump comprises:
a substrate;
a first chamber formed on the substrate by etching;
a resonance board, wherein a hollow hole and a movable portion are formed on the resonance board by etching, and the resonance board is stacked on the substrate; the movable portion is a flexible structure formed by a portion of the resonance board which is not fixedly disposed on the substrate;
a spacing layer coated and stacked on portions of the resonance board except the movable portion;
an actuation board, wherein a suspension portion, an outer frame portion, and a plurality of brackets are formed on the actuation board by etching, and the actuation board is stacked on the spacing layer; the suspension portion is connected to and suspended from the outer frame portion, and the suspension portion is supported by the outer frame portion through the brackets; wherein a plurality of through holes is provided between the suspension portion, the outer frame portion, and the brackets for gas flowing therethrough, and a second chamber is defined between the actuation board and the resonance board;
a piezoelectric element coated and stacked on the suspension portion of the actuation board; and
an outlet board provided with a third chamber and an outlet hole by etching, wherein the outlet board is stacked on the outer frame portion of the actuation board, so that the third chamber is corresponding to the suspension portion of the actuation board, and the outlet hole is in communication with the third chamber; and
an inlet board provided with at least one inlet hole by etching, wherein the inlet board is stacked below the substrate;
wherein the actuation board is driven by the piezoelectric element to move reciprocatingly so as to draw the gas into the first chamber through the inlet hole and pass through the hollow hole of the resonance board, thereby a resonance effect is generated between the actuation board and the movable portion of the resonance board for gas transmission.
